SEQUENCE LISTING (7 sequences)

SEQ ID NO: 1 - DNA, 24 bp, Artificial
  Synthetic sequence or genetically engineered sequence that does not naturally occur
tgagtcaaaactagagcctggacc

SEQ ID NO: 2 - DNA, 23 bp, Artificial
  Synthetic sequence or genetically engineered sequence that does not naturally occur
agttcgcttctcgcttctgttcg

SEQ ID NO: 3 - DNA, 24 bp, Artificial
  Synthetic sequence or genetically engineered sequence that does not naturally occur
aaattggactaatcgataccgtcg

SEQ ID NO: 4 - DNA, 23 bp, Artificial
  Synthetic sequence or genetically engineered sequence that does not naturally occur
gagtgattgactacccgtcagcg

SEQ ID NO: 5 - DNA, 590 bp, Artificial
  Synthetic sequence or genetically engineered sequence that does not naturally occur
tgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttca

SEQ ID NO: 6 - DNA, 194 bp, Artificial
  Synthetic sequence or genetically engineered sequence that does not naturally occur
tgaaagaccccacctgtaggtttggcaagctagcacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttca

SEQ ID NO: 7 - DNA, 5174 bp, Artificial
  Synthetic sequence or genetically engineered sequence that does not naturally occur
tgaaagacccccgctgacgggtagtcaatcactcagaggagaccctcccaaggaacagcgagaccacaagtcggatgcaactgcaagagggtttattggatacacgggtacccgggcgactcagtcaatcggaggactggcgcgccgagtgaggggttgtgggctcttttattgagctcggggagcagaagcgcgcgaacagaagcgagaagcgaactgattggttagttcaaataaggcacagggtcatttcaggtccttggggcaccctggaaacatctgatggttctctagaaactgctgagggctggaccgcatctggggaccatctgttcttggccctgagccggggcaggaactgcttaccacagatatcctgtttggcccatattcagctgttccatctgttcttggccctgagccggggcaggaactgcttaccacagatatcctgtttggcccatattcagctgttccatctgttcctgaccttgatctgaacttctctattctcagttatgtatttttccatgccttgcaaaatggcgttacttaagctagcttgccaaacctacaggtggggtctttcattcccccctttttctggagactaaataaaatcttttattttatctatggctcgtactctataggcttcagctggtgatattgttgagtcaaaactagagcctggaccactgatatcctgtctttaacaaattggactaatcgataccgtcgatcgacctcgacagatcttaagccagttttcgtacccttgactgcgtttcatcgattcgctactaacattgccttttcctccttccctcccacaggtggaagagcaagctttgatgagccgccaccatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggatcgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatcaattctctagagctcgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgaggccggccctgcgactctagaggatctgcgactctagaggatcataatcagccataccacatttgtagaggttttacttgctttaaaaaacctcccacacctccccctgaacctgaaacataaaatgaatgcaattgttgttgttaacttgtttgttgcagcttataatggttacaaataaagcaatagcatcacaaatttcacaaataaagcatttttttcactgcattctagttgtggtttgtccaaactcatcaatgtatcttatcatgtctggatctgcgactctagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgcgactctagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatccccgggtaccgagctcgaaggccggccgtttaaaccaatcgaattcccgcggctagacccagctttcggaagttcctattcggaagttcctattctctagaaagtataggaacttctcgatatggtcgatcgacctgcaggaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcaggcggccgcgaattcactagtgattgcagcgtacggatccgccgccgccatggctccggtaggtccagagtcttcagagatcaagtcccaccttccaagtcctggcatctcacgacgtctggggagctacctgcattaagtcagaactgaggtgggtttgggctgaggtagagcctgggcagaggccataattacttcttgtggaactctcaaaggtcggacaggaagcatggctggttccatatatctactgcctcgaatcgatgaattcgagctcggtacccggggatcgaagttcctattcggaagttcctattctctagaaagtataggaacttctcgacctgcaggcatgcaagctgggggggtcgacgtcgagaaggagtgagggctggataaagggaggatcgaggcggggtcgaacgaggaggttcaagggggagagacggggcggatggaggaagaggaggcggaggcttagggtgtacaaagggcttgacccagggaggggggtcaaaagccaaggcttcccaggtcacgatgtaggggacctggtctgggtgtccatgcgggccaggtgaaaagaccttgatcttaacctgggtgatgaggtctcggttaaaggtgccgtctcgcggccatccgacgttaaaggttggccattctgcagagcagaaggtaacccaacgtctcttcttgacatctaccgactggttgtgagcgatccgctcgacatctttccagtgacctaaggtcaaacttaagggagtggtaacagtctggcccatattttcagacaaatacagaaacacagtcagacagagacaacacagaacgatgctgcagcagacaagacgcgcggcgcggcttcggtcccaaaccgaaagcaaaaattcagacggaggcgggaactgttttaggttctcgtctcctaccagaaccacatatccctcctctaaggggggtgcaccaaagagtccaaaacgatcgggatttttggactcaggtcgggccacaaaaacggcccccgaagtccctgggacgtctcccagggttgcggccgggtgttccgaactcgtcagttccaccacgggtccgccagatacagagctagttagctaactagtaccgacgcaggcgcataaaatcagtcatagacactagacaatcggacagacacagataagttgctggccagcttacctcccggtggtgggtcggtggtccctgggcaggggtctcccgatcccggacgagcccccaaatgaaagacccccgctgacgggtagtcaatcactcagaggagaccctcccaaggaacagcgagaccacaagtcggatgcaactgcaagagggtttattggatacacgggtacccgggcgactcagtcaatcggaggactggcgcgccgagtgaggggttgtgggctcttttattgagctcggggagcagaagcgcgcgaacagaagcgagaagcgaactgattggttagttcaaataaggcacagggtcatttcaggtccttggggcaccctggaaacatctgatggttctctagaaactgctgagggctggaccgcatctggggaccatctgttcttggccctgagccggggcaggaactgcttaccacagatatcctgtttggcccatattcagctgttccatctgttcttggccctgagccggggcaggaactgcttaccacagatatcctgtttggcccatattcagctgttccatctgttcctgaccttgatctgaacttctctattctcagttatgtatttttccatgccttgcaaaatggcgttacttaagctagcttgccaaacctacaggtggggtctttca

(19) United States
(12) Patent Application Publication
(10) Pub. No.: US 2005/0118710 A1
(43) Pub. Date: Jun. 2, 2005

(54) Methods and compositions for defining gene function

(76) Inventors: Gwenn Hansen, Spring, TX (US); Alejandro Abuin, The Woodlands, TX (US); Brian Zambrowicz, The Woodlands, TX (US); Carl Johan Friddle, The Woodlands, TX (US); William Dempsey, The Woodlands, TX (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 10/954,645

(22) Filed: Sep. 30, 2004

Related U.S. Application Data
(60) Provisional application No. 60/507,437, filed on Sep. 30, 2003.

(51) Int. Cl. C12N 15/85

(52) U.S. Cl. 435/325; 435/455

(57) ABSTRACT

A process for producing and analyzing insertionally mutated cell clones is provided. Cells incorporating insertional mutations are provided. A collection of insertionally mutated cell clones is also provided.

The present application claims the benefit of U.S. Provisional Application No. 60/507,437, filed Sep. 30, 2003, which is incorporated herein by reference for any purpose.

### 1.0. FIELD

Methods for generating and characterizing insertional mutations in cells are provided. Cells stably incorporating insertional mutations are also provided.

### 2.0. BACKGROUND

Many human therapeutics (with certain exceptions, including, e.g., antibiotics that directly target the pathogen) directly or indirectly interact with a product or element encoded by a finite set of genomic sequence information. Consequently, scientific scrutiny has turned to defining that portion of the encoded sequence information that may present a clear path for medical intervention. Many therapeutic products act by modulating physiology. Of the thousands of biochemically and structurally related products encoded within the human genome, the scientific community has clearly defined the physiological roles of only a fraction of these products. For a variety of reasons, in certain instances, the mouse has emerged as a model organism for characterizing-by-proxy the physiological significance of human biological sequences. As a mammal, the mouse shares many of the major organ systems of humans and often modulates the functions of these organ systems using orthologous products. Furthermore, in certain instances, the mouse also allows for the genetic engineering of its genome.

In certain instances, mouse embryonic stem (ES) cell technology provides an approach for chromosome engineering and, consequently, the direct testing of genomic hypotheses.

### 3.0. SUMMARY

In certain embodiments, a process for producing a collection of individually characterized insertionally mutated mammalian cell clones is provided. In certain embodiments, the process comprises infecting mammalian cells with a retroviral gene trap construct. In certain embodiments, the process further comprises selecting mammalian cell clones stably incorporating an integrated proviral form of said retroviral gene trap construct. In certain embodiments, the process further comprises identifying in vitro a region of genomic DNA adjacent to the integrated proviral form of said retroviral gene trap construct.

In certain embodiments, the mammalian cells are infected with a retroviral gene trap construct at a multiplicity of infection (M.O.I.) of less than 5. In certain embodiments, the multiplicity of infection is less than 1. In certain embodiments, the multiplicity of infection is less than 0.5.

In certain embodiments, the identifying comprises an inverse polymerase chain reaction (IPCR). In certain embodiments, the inverse polymerase chain reaction comprises at least one polymerase selected from Pfu, Taq, Isis, Vent, Pwo, Phusion, and Tth. In certain embodiments, the identifying is by sequencing at least 50 bases of genomic DNA adjacent to the integrated proviral form of said retroviral gene trap construct. In certain embodiments, the identifying does not involve a reverse transcriptase reaction.

In certain embodiments, a collection of at least 10,000 different mammalian cell clones is selected. In certain embodiments, the collection of at least 10,000 different mammalian cell clones comprises at least 10,000 different mammalian cell cones that each have an integrated proviral form of said retroviral gene trap construct in a different gene is selected.

In certain embodiments, a collection of individually characterized insertionally mutated mammalian cell clones is provided.

### 4.0. BRIEF DESCRIPTION OF THE FIGURES

FIG. 1 shows a schematic representation of an inverse polymerase chain reaction (IPCR).

FIG. 2 shows a schematic representation of VICTR 48. “LTR” is a retroviral long terminal repeat. “SA” is a splice acceptor site. “NEO” is a neomycin resistance gene. “pA” is a polyadenylation site. “SV40tpA” is the SV40 triple polyadenylation sequence. “PGK” is a PGK promoter. “BTK” and “SD” are the first exon and the splice donor site of the mouse BTK gene. The splice donor site is followed by a portion of the first intron of the BTK gene. Restriction sites are indicated. An asterisk (*) after the restriction site name indicates that it is a unique site on the construct.

FIG. 3 shows exemplary products from an IPCR analysis of several gene-trapped ES cell clones, as discussed in Example 6.2.

FIG. 4 shows the sequence of a Moloney murine leukemia virus long terminal repeat (LTR), lacking at least a portion of the enhancer region (SEQ ID NO:5).

FIG. 5 shows a modified LTR, which lacks at least a portion of the enhancer region, and also lacks a cryptic splice donor within the LTR (SEQ ID NO:6).

FIG. 6A-C shows the sequence of VICTR 48 (SEQ ID NO: 7).

### 5.0. DETAILED DESCRIPTION

The section headings used herein are for organizational purposes only and are not to be construed as limiting the subject matter described. All references cited in this application are expressly incorporated by reference herein for any purpose.

In this application, the use of the singular includes the plural unless specifically stated otherwise. In this application, the use of “or” means “and/or” unless stated otherwise. Furthermore, the use of the term “including”, as well as other forms, such as “includes” and “included”, is not limiting. In various embodiments, standard techniques may be used for recombinant DNA, oligonucleotide synthesis, tissue culture, transformation and transfection. In various embodiments, enzymatic reactions and purification techniques may be performed according to manufacturer's specifications or as commonly accomplished in the art or as described herein. In various embodiments, techniques and procedures may be generally performed according to conventional methods known in the art and as described in various general and more specific references that are cited and discussed throughout the present specification and/or that are known to one skilled in the art. See e.g., Sambrook et al. Molecular Cloning: A Laboratory Manual (2d ed., Cold Spring Harbor Laboratory Press, Cold Spring Harbor, N.Y. (1989).

In certain embodiments, a process for defining the physiological role of genetically encoded biological sequences (including, but not limited to, proteins, polypeptides, amino acid sequences, polynucleotide and nucleotide sequences) within the context of mammalian biology is provided. In certain embodiments, methods are provided for culturing and processing of eukaryotic cells such that genetically engineered eukaryotic cell clones having defined genomic insertions/mutations are generated. Eukaryotic cells for use in the methods, in various embodiments, include but are not limited to insect cells (including but not limited to Drosophila melanogaster cells), C. elegans cells, rodent cells (including but not limited to mouse cells, rat cells, and hamster cells), chicken cells, primate cells (including but not limited to monkey cells and human cells. In certain embodiments, the cells are genetically engineered by nonspecific insertional mutation. In certain embodiments, methods are provided for culturing and processing of mouse ES cells such that genetically engineered ES cell clones having defined genomic insertions/mutations are generated.

In certain embodiments, the method does not include certain expensive and/or time-consuming processing steps. In certain embodiments, the method may be suitable for the commercial scale production of mutated eukaryotic cells, including but not limited to, mutated mouse ES cells.

Certain embodiments relate to processes for culturing, generating, and characterizing mutated eukaryotic cells, including but not limited to mouse ES cells. In certain embodiments, the mutated eukaryotic cells can be used to produce organisms capable of germline transmission of the insertionally mutated allele. In certain embodiments, mutated mouse ES cells are used to produce mice capable of germline transmission of the insertionally mutated allele.

In certain embodiments, gene trapping is a method of random insertional mutagenesis that uses DNA as a mutagen. In certain embodiments, the DNA is initially introduced as a retrovirus, which is reverse transcribed in the cell to produce a DNA provirus, which acts as the mutagen. In certain embodiments, a portion of the DNA encodes a selectable marker. In certain embodiments, a gene trap construct integrates into an intron or an exon of a gene. In certain embodiments, gene trap constructs are designed to preferentially integrate into introns and/or exons. In various embodiments, after integration into an intron or exon, the cellular splicing machinery splices construct-encoded sequences to one or more endogenous sequences that are co-transcribed on the same mRNA. In certain embodiments, a gene trap construct contains a sequence encoding a selectable marker. In certain embodiments, the sequence encoding the selectable marker is preceded by a splice acceptor sequence. In certain embodiments, the sequence encoding the selectable marker is not preceded by a promoter. In certain embodiments, the cellular splicing machinery splices endogenous sequence from the trapped gene onto the 5′ end of the sequence encoding the selectable marker. In certain embodiments, the selectable marker is expressed only if the gene-trap construct encoding the selectable marker has integrated into an intron. In certain embodiments, the selectable marker is expressed only if the gene-trap construct encoding the selectable marker has integrated into an exon. In certain embodiments, e.g., when the selectable marker gene encodes antibiotic resistance, cells that have the gene trap construct integrated in their genomes in such a way that the selectable marker is expressed can be selected in culture.

Exemplary insertional mutagenesis of eukaryotic cells is described, e.g., in Friedrich and Soriano, 1991, Genes Dev. 5(9):1513-23; Friedrich and Soriano, 1993, Methods Enzymol. 1993;225:681-701; PCT Publication Nos. WO 98/14614; WO 99/07389; WO 99/50426; and WO 00/31236; and U.S. Pat. Nos. 5,364,783; 6,303,327; 6,080,567; 6,136,566; 6,207,371; 6,228,639; 6,436,707; 6,776,988; WO 00/31236; and U.S. Pat. No. 6,139,833; each of which is incorporated by reference herein in its entirety for any purpose. Certain constructs described in the cited publications and patents may be suitable for practicing certain embodiments of the methods described herein. In certain embodiments, one or more constructs described in PCT Publication Nos. WO 98/14614; WO 99/07389; WO 99/50426; and WO 00/31236; and U.S. Pat. Nos. 5,364,783; 6,303,327; 6,080,567; 6,136,566; 6,207,371; 6,228,639; 6,436,707; 6,776,988; WO 00/31236; and U.S. Pat. No. 6,139,833 are used in the methods described herein.

In certain instances, a construct may be modified before being used for certain methods described herein. Thus, in certain embodiments, one or more structural features of a construct may be omitted or modified before it is used in certain embodiments of the methods described herein. In certain embodiments, one skilled in the art can modify a particular construct for use in certain methods described herein.

Certain methods of using genetically engineered mouse ES cells to produce chimeric mice capable of germ line transmission of genetically engineered alleles are known (see, e.g., inter alia, U.S. Pat. Nos. 6,204,061; 5,789,215; and U.S. Pat. No. 6,087,555; each of which is incorporated by reference herein in its entirety for any purpose). In certain embodiments, mouse ES cells are co-cultured with feeder cells that have been engineered to express leukocyte inhibitory factor (LIF). In certain embodiments, by co-culturing with such feeder cells, one may avoid incurring the additional expense of making and/or purchasing exogenous LIF to be added to the culture.

In certain embodiments, the presence of an internal ribosome entry site (IRES) sequence operably positioned upstream from a selectable marker of a gene-trap construct increases the chance of selecting a mutated cell clone that has not integrated the gene-trap construct into the coding region of a gene. In certain embodiments, integrating into the coding region of a gene (i.e., integrating into introns or exons located downstream from the initiation codon and upstream from the stop codon) is a feature of a gene trap event that alters or disrupts the function of the gene. Thus, in certain embodiments, the presence of an IRES element functionally situated upstream from a gene trap construct encoded selectable marker is not desirable.

As used herein, a selectable marker is a marker that provides a way of identifying cells that contain the gene encoding the marker. Selectable markers include, but are not limited to, antibiotic resistance markers, light producing markers, and fluorescent markers. In certain embodiments, the construct will lack an IRES operatively positioned upstream from the selectable marker.

In certain instances, the presence of a cryptic splice donor (SD) site in reverse-orientation within a retroviral long terminal repeat (LTR) can increase the likelihood of selecting a mutated cell clone that has incorporated the proviral gene-trap construct outside of the protein-encoding region of the gene. In certain instances, certain vectors derived from Moloney murine leukemia virus (MLV) can increase the likelihood of selecting a mutated cell clone that has incorporated the proviral gene-trap construct outside of the protein-encoding region of the gene. In certain instances, the reverse-oriented SD site can be spliced by the cellular splicing machinery to a splice acceptor (SA) site that is operatively positioned upstream from a selectable marker in the gene-trap construct. Accordingly, in certain embodiments, gene trap constructs are engineered to lack a SD site operatively positioned upstream from a SA site that is operatively positioned upstream from a selectable marker.

In certain instances, gene trapped ES cell clones are identified and catalogued using products from either 3′- or 5′-rapid amplification of cDNA ends (RACE). In certain instances, polyadenylated mRNA is isolated from an ES cell sample (in certain instances, the number of ES cells present in a “confluent” well from a 96 well microtiter plate), reverse transcribed, and a nested set of primers are employed in the polymerase chain reaction to produce a template that is subsequently sequenced. In certain instances, given the inherent difficulties of working with small samples of RNA, such operations may operate at or near practical levels of detection. Thus, in certain instances, when RACE-type procedures are adapted for robotic “high throughput” processing, yet more sensitivity is exchanged for higher throughput.

In certain embodiments, methods of identifying a gene that has been insertionally mutated by a gene trap construct are provided. In certain embodiments, the gene has been insertionally mutated using an engineered retrovirus. In certain embodiments, the gene has been insertionally mutated using a DNA gene trap construct. In certain embodiments, by using genomic DNA as the template for inverse PCR amplification, RNA “capture” (e.g., enrichment or isolation of RNA) and/or reverse transcription is not used. For example, automation of 3′ RACE reactions may involve a solid phase RNA capture method that utilizes 96 well microtiter plates derivatized with an oligo dT moiety (to “capture” polyA RNA). Such “custom” microtiter plates may be expensive and perishable. Additionally, in certain instances, reverse transcriptase may also be expensive and perishable and therefore undesirable for a high-throughput assay. Accordingly, in certain embodiments, the methods described herein include a process for the high-throughput analysis of a collection or library, of gene trapped eukaryotic cell clones, including but not limited to gene-trapped ES cell clones. In certain embodiments, the method does not include the selective enrichment or “capture” of RNA from the eukaryotic cell clones (e.g., using RNA “isolation,” including RNA enrichment methods such as, for example, the use of oligo-dT to bind polyadenylated mRNA). In certain embodiments, the process does not include the use of reverse transcriptase to prepare templates for sequencing and/or for identifying endogenous exon sequences that flank the integrated gene trap construct.

In certain embodiments, inverse PCR (IPCR) is used for high-throughput analysis of gene-trapped cells. Exemplary IPCR is described, e.g., in Ochman et al., Genetics 120: 621-623 (1988); Hui et al., Methods Mol. Biol. 192: 249-274 (2002); Hui et al., Cell Mol. Life Sci. 54: 1403-1411 (1998); Benkel et al. Genet. Anal. 13: 123-127 (1996); Offringa et al. Methods Mol. Biol. 49: 181-195 (1995); and Garces et al. Methods Mol. Biol. 161: 3-8 (2001); and references cited therein; each of which is incorporated by reference herein for any purpose.

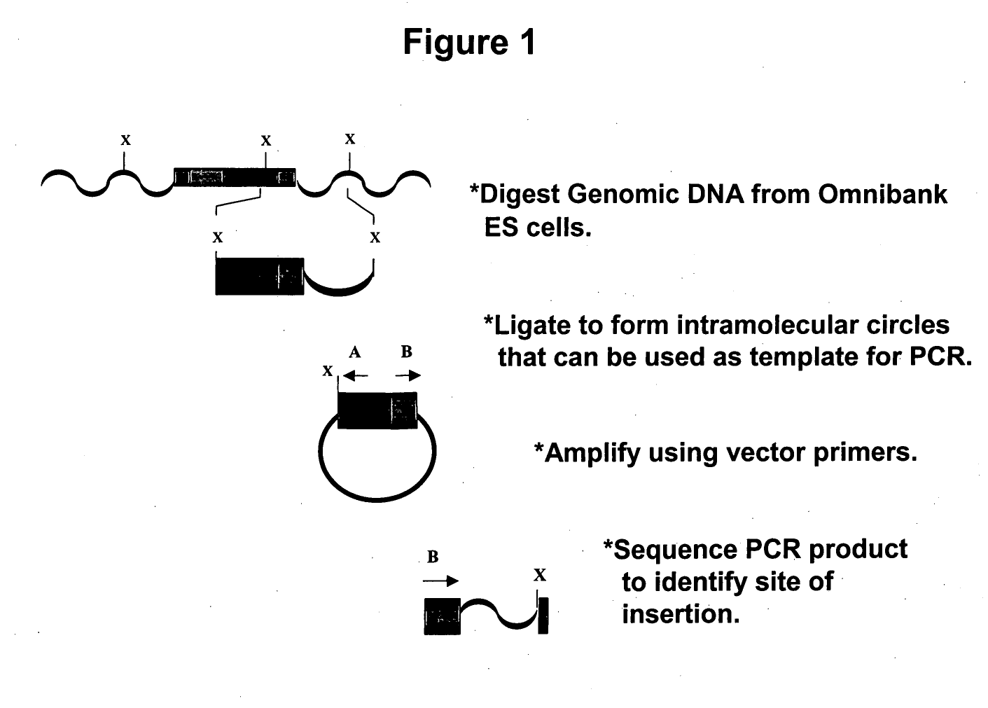

A schematic representation of an exemplary IPCR is shown in FIG. 1. Genomic DNA from a gene-trapped cell is isolated and digested with a restriction enzyme, X. After digestion, the genomic DNA is ligated to form intramolecular circles. The ligated genomic DNA is then subjected to a PCR reaction in the presence of two primers, A and B. Primers A and B anneal to gene trap construct sequences such that the PCR reaction amplifies genomic sequence that is located between the annealing sites for primers A and B on the intramolecular circle. The result of the PCR reaction is a linear DNA containing a genomic sequence that was adjacent to the gene-trap construct in the cell's genomic DNA, flanked by construct sequences that anneal to primers A and B. One or both of primers A and B can then be used to sequence the genomic DNA. Alternatively, one or more different primers can be used, in addition to or in place of primers A and B, to sequence the genomic sequence in the amplified DNA. The sequence of the genomic DNA can then be used in a BLAST search to identify the gene into which the gene-trap target has integrated in the gene-trapped cell.

In certain embodiments, an IPCR reaction comprises at least one polymerase. In certain embodiments, an IPCR reaction comprises at least two polymerases. In certain embodiments, an IPCR reaction comprises at least three polymerases. In certain embodiments, at least one polymerase present in an IPCR reaction is a thermostable polymerase. In various embodiments, polymerases that can be used in IPCR reactions include, but are not limited to, Pfu, Taq, Isis, Vent, Pwo, Phusion, and Tth. In certain embodiments, 0.005 to 1 units per μl of a polymerase are used in an IPCR reaction. In certain embodiments, 0.01 to 0.5 units per μl of a polymerase are used in an IPCR reaction. In certain embodiments, 0.01 to 0.1 units per μl of a polymerase are used in an IPCR reaction. In certain embodiments, units are defined according to the polymerase manufacturer's definition.

In certain embodiments, primers that anneal to gene-trap construct sequences are selected for IPCR. In certain embodiments, one primer is selected to anneal to a gene-trap construct sequence adjacent to a selected restriction enzyme cut site. In certain embodiments, a second primer is selected to anneal to an end of the gene-trap construct such that it is predicted to anneal adjacent to the genomic DNA into which the gene-trap construct has integrated. Primers are selected, in certain embodiments, such that they will both anneal to the same contiguous piece of DNA following restriction enzyme digestion. Furthermore, in certain embodiments, two primers are selected such that, following ligation of the digested DNA into intramolecular circles, the primers will be extended in opposite directions around the circle.

In certain embodiments, a primer is selected such that when it anneals, the 3′ end of the primer is within 100 bases of a selected restriction enzyme cut site. In certain embodiments, a primer is selected such that when it anneals, the 3′ end of the primer is within 50 bases of a selected restriction enzyme cut site. In certain embodiments, a primer is selected such that when it anneals, the 3′ end of the primer is within 20 bases of a selected restriction enzyme cut site. In certain embodiments, a primer is selected such that when it anneals, the 3′ end of the primer is within 10 bases of a selected restriction enzyme cut site. In certain embodiments, a primer is selected such that when it anneals, the 3′ end of the primer is more than 100 bases from a selected restriction enzyme cut site. One skilled in the art can select an appropriate primer length and sequence for PCR.

In certain embodiments, a primer is selected such that when it anneals, the 3′ end of the primer is within 100 bases of the end of the gene-trap construct when integrated into a cell genome. In certain embodiments, a primer is selected such that when it anneals, the 3′ end of the primer is within 50 bases of the end of the gene-trap construct when integrated into a cell genome. In certain embodiments, a primer is selected such that when it anneals, the 3′ end of the primer is within 20 bases of the end of the gene-trap construct when integrated into a cell genome. In certain embodiments, a primer is selected such that when it anneals, the 3′ end of the primer is within 10 bases of the end of the gene-trap construct when integrated into a cell genome. In certain embodiments, a primer is selected such that when it anneals, the 3′ end of the primer is more than 100 bases from the end of the gene-trap construct when integrated into a cell genome. One skilled in the art can select an appropriate primer length and sequence for PCR.

In certain embodiments, using inverse PCR (IPCR) to analyze the genomic sequence flanking the integration site of the gene trap construct provides sufficient sensitivity such that the procedure requires less starting material than RACE-based methods of identifying one or more flanking exons. For example, in certain embodiments, less than 2 percent of the clonal cells present in a confluent well of 96 well microtiter plate are used to generate a template for identifying one or more flanking regions of genomic DNA. In certain embodiments, less than 5 percent of the clonal cells present in a confluent well of 96 well microtiter plate are used. In certain embodiments, less than 10 percent of the clonal cells present in a confluent well of 96 well microtiter plate are used. In certain embodiments, less than 20 percent of the clonal cells present in a confluent well of 96 well microtiter plate are used. In certain embodiments, less than 30 percent of the clonal cells present in a confluent well of 96 well microtiter plate are used. In certain embodiments, less than 40 percent of the clonal cells present in a confluent well of 96 well microtiter plate are used. In certain embodiments, less than 50 percent of the clonal cells present in a confluent well of 96 well microtiter plate are used. In certain embodiments, less than 60 percent of the clonal cells present in a confluent well of 96 well microtiter plate are used. In certain embodiments, less than 70 percent of the clonal cells present in a confluent well of 96 well microtiter plate are used. In certain embodiments, less than 80 percent of the clonal cells present in a confluent well of 96 well microtiter plate are used. In certain embodiments, less than 90 percent of the clonal cells present in a confluent well of 96 well microtiter plate are used. In certain embodiments, identification of one or more flanking regions of genomic DNA involves sequencing the template and comparing its sequence with known genomic sequence data.

In certain embodiments, to facilitate identification of the integration site of the gene-trap construct, at least about 35 bases of genomic DNA from a region flanking the integration site is sequenced. In certain embodiments, at least about 40 bases of genomic DNA from a region flanking the integration site is sequenced. In certain embodiments, at least about 45 bases of genomic DNA from a region flanking the integration site is sequenced. In certain embodiments, at least about 50 bases of genomic DNA from a region flanking the integration site is sequenced. In certain embodiments, at least about 70 bases of genomic DNA from a region flanking the integration site is sequenced. In certain embodiments, at least about 85 bases of genomic DNA from a region flanking the integration site is sequenced. In certain embodiments, at least about 100 bases of genomic DNA from a region flanking the integration site is sequenced. In certain embodiments, at least about 150 bases of genomic DNA from a region flanking the integration site is sequenced. In certain embodiments, at least about 200 bases of genomic DNA from a region flanking the integration site is sequenced. In certain embodiments, at least about 250 bases of genomic DNA from a region flanking the integration site is sequenced. In certain embodiments, at least about 350 bases of genomic DNA from a region flanking the integration site is sequenced. In certain embodiments, at least about 450 bases of genomic DNA from a region flanking the integration site is sequenced. In certain embodiments, at least about 500 bases or more of genomic DNA from a region flanking the integration site is sequenced.

In certain instances, RACE-based methods enrich for spliced exon sequence far upstream (for 5′ RACE), or downstream (for 3′ RACE) from the gene trap construct insertion site. In certain embodiments, using the methods described herein, far upstream or far downstream exon sequences are less enriched as compared to certain RACE-based methods.

In certain embodiments, IPCR-mediated sequencing of genomic flanking DNA provides enhanced sequencing sensitivity. Thus, in certain embodiments, fewer cells can be used to produce sufficient genomic DNA for IPCR, which leaves cells available for other uses and/or for one or more additional IPCR reactions. In certain embodiments, a single well of a 96 well plate contains enough cells for both IPCR and at least one other use, which may include one or more additional IPCR reactions. In certain embodiments, only ⅓ of the cells in a 96 well plate are used to produce genomic DNA for IPCR, leaving ⅔ of the cells for at least one other use. In certain embodiments, only ¼ of the cells in a 96 well plate are used to produce genomic DNA for IPCR, leaving ¾ of the cells for at least one other use. In certain embodiments, only ⅕ of the cells in a 96 well plate are used to produce genomic DNA for IPCR, leaving ⅘ of the cells for at least one other use. In certain embodiments, only 1/10 of the cells in a 96 well plate are used to produce genomic DNA for IPCR, leaving 9/10 of the cells for at least one other use.

In certain embodiments, using IPCR to amplify genomic flanking sequence provides a product that is suitable for sequencing. In certain instances, a template generated from a RACE reaction requires a “clean-up” step that involves running the template through a size exclusion chromatography column prior to sequencing. In contrast, in certain embodiments, a template generated by IPCR does not require chromatography prior to initiating a sequencing reaction.

In certain embodiments, IPCR methods allow the use of higher density plate formats, including, but not limited to, 384 well plates and other high density plate formats. As used herein, “high density plate formats” include plates having more wells per plate area than a 96-well plate. In certain embodiments, by using high density plate formats, lower reaction volumes are needed. In certain embodiments, using high density plate formats enhances automated performance, e.g., by increasing the rate of throughput and/or by decreasing the amount of reagents used per sample analyzed. In various embodiments, transfer of a portion of the gene-trapped cells to, for example, a 384 well format during splitting provides efficiencies by lowering the volume of the cultures, decreasing the amount of reagents required for each reaction, and/or decreasing the number of plates that are handled and processed to define the genomic insertion site of the gene trap construct within the cell clones.

In certain embodiments, genomic DNA is obtained from gene-trapped cell clones grown in a 96 well format. In certain embodiments, cells grown in a 96 well format can be used when larger quantities of genomic DNA are desired. In certain embodiments, one or more of certain subsequent reactions, including, but not limited to, restriction digestion, ligation, PCR amplification, and sequencing, can be performed in a higher density plate format (e.g., a 384 well plate or other high density format). In certain embodiments, standard plate formats will be employed so that certain standard automated/robotic plate and fluid handling devices can be used during processing (such devices include, but not limited to, a Beckman Coulter Biomek FX, a Packard mini track, etc., and updated or related variants thereof).

In certain embodiments, certain restriction enzymes, and/or certain combinations of restriction enzymes provide enhanced yields of IPCR-amplified template. In certain embodiments, the same pair of construct-specific primers can be used for two or more different constructs. Thus, in certain embodiments, the same pair of construct-specific primers can be used to prime an IPCR reaction of circularized template from first cells that have been gene-trapped with a first construct and to prime an IPCR reaction of circularized template from second cells that have been gene-trapped with a second construct. In certain embodiments, the same pair of construct-specific primers can be used to prime an IPCR reaction of circularized template created using a first restriction enzyme or combination of restriction enzymes and can also be used to prime an IPCR reaction of circularized template created using a second restriction enzyme or combination of restriction enzymes.

In certain embodiments, the genomic DNA from gene-trapped cells is digested with one restriction enzyme. In certain embodiments, the genomic DNA from gene-trapped cells is digested with at least two different restriction enzymes in the same reaction. In certain embodiments, the genomic DNA from gene-trapped cells is digested with at least three different restriction enzymes in the same reaction. In certain embodiments, the genomic DNA from gene-trapped cells is digested with at least four different restriction enzymes in the same reaction. In certain embodiments, the genomic DNA from gene-trapped cells is digested with at least five different restriction enzymes in the same reaction. In certain embodiments, the genomic DNA from gene-trapped cells is digested with at least six different restriction enzymes in the same reaction.

In certain embodiments, the gene-trap construct that is integrated into the genomic DNA is not cut in the digestion reaction. In certain embodiments, the gene trap construct that is integrated into the genomic DNA is cut at one site in the digestion reaction. In certain embodiments, the gene trap construct that is integrated into the genomic DNA is cut at two sites in the digestion reaction. In certain embodiments, the gene trap construct that is integrated into the genomic DNA is cut at three sites in the digestion reaction. In certain embodiments, the gene trap construct that is integrated into the genomic DNA is cut at four or more sites in the digestion reaction.

In certain embodiments, genomic DNA from gene-trapped cells is subjected to at least two separate reactions, wherein no two reactions contain the same combination of restriction enzymes. In certain embodiments, genomic DNA from gene-trapped cells is subjected to at least three separate reactions, wherein no two reactions contain the same combination of restriction enzymes. In certain embodiments, genomic DNA from gene-trapped cells is subjected to at least four separate reactions, wherein no two reactions contain the same combination of restriction enzymes.

In certain embodiments, a multiple cloning site (MCS) is incorporated into the gene-trap construct. In certain embodiments, a particular restriction enzyme will cut the construct at one or more locations. In certain embodiments, a particular restriction enzyme will cut the construct in the MCS and in at least one location outside of the MCS. In certain embodiments, sufficient gene-trap construct sequence is maintained after digestion to allow priming by oligos for IPCR. In certain embodiments, two or more restriction enzymes may cut the gene-trap construct at locations close to one another. In certain embodiments, the same pair of construct-specific primers can be used to prime IPCR reactions of circularized templates from genomic DNA that has been digested with different enzymes.

In certain embodiments, the gene-trap construct includes one or more sites for a first restriction enzyme that leaves a “sticky” end that is compatible with the sticky end left by a second restriction enzyme that also cuts the gene-trap construct at one or more sites. In certain embodiments, three or more restriction enzymes leave the same compatible sticky ends. As used herein, a sticky end refers to an overhang of at least one nucleotide left after cleavage of DNA with a restriction enzyme. The overhang may be either a 5′ overhang or a 3′ overhang. In certain embodiments, a two nucleotide overhang is left after cleavage. In certain embodiments, a three nucleotide overhang is left after cleavage. In certain embodiments, a four nucleotide overhang is left after cleavage. In certain embodiments, an overhang having more than four nucleotides is left after cleavage. Examples of certain groups of restriction enzymes that leave compatible sticky ends are known in the art (see, e.g., the New England Biolabs 2003 catalog, Beverly, Mass.). Exemplary groups of restriction enzymes that leave compatible sticky ends include, but are not limited to, BglII, BamHI, BclI, and BstYI; EcoRI, MfeI, and ApoI; PstI, NsiI, and SbfI; ApaLI and SfcI; NcoI, BspHI, RcaI, and PciI; SpeI, NheI, XbaI, and AvrII; Acc65I, BsiWI, and BsrGI; AclI, ClaI, BstBI, HinPlI, HpaII, and NarI; AgeI, XmaI, BsaWI, and BspEI; MluI, AscI, and BssHI; AscI, NdeI, MseI, and BfaI; PvuI, PacI, and AsiSI; EaeI, EagI, and NotI; XhoI, PspXI, and SalI. One skilled in the art can identify additional members of the exemplary groups and/or additional groups of restriction enzymes that leave compatible sticky ends. In certain embodiments, fewer than all members of a group are used in a reaction.

In certain embodiments, a gene-trap construct includes one or more sites for a restriction enzyme that leaves blunt ends (i.e., a restriction enzyme that does not leave overhangs). In certain embodiments, a gene-trap construct includes at least one site for each of at least two different restriction enzymes that leave blunt ends. In certain embodiments, one or more different restriction enzymes that leave blunt ends are used in a reaction. Exemplary restriction enzymes that leave blunt ends include, but are not limited to, FspI, HincII, EcoRV, HpaI, MscI, NaeI, NruI, PvuII, ScaI, SfoI, SmaI, SnaBi, and StuI.

In certain embodiments, a single copy of the gene trap construct is incorporated into the genome of a cell. In this manner, in certain embodiments where the mutation caused by insertion of the gene-trap construct exerts a dominant negative effect, the observed phenotype can be associated with the gene trapped allele. Similarly, in certain embodiments, the observed phenotype can be associated with the gene-trapped allele when the mutation caused by insertion of the gene-trap construct is present in the homozygous state (e.g., a sex chromosome has been mutated and/or the cell has been further manipulated to produce a cell homozygous for the gene trapped allele). In certain embodiments, to enrich the number of gene-trapped cell clones having just one gene-trap construct incorporated into their genomes, the multiplicity of infection (m.o.i.) is reduced. In certain embodiments, a m.o.i. of 0.3 (virus:cell) is predicted (with about 95% certainty) to yield about 95% cells that contain a single gene trap event, after selection for cells that have at least one gene-trap construct incorporated into their genomes. In certain embodiments, packaged preparations of retroviral gene trap constructs are tested for their ability to confer expression of a construct-encoded selectable marker to ES cells and a viral titer is determined. In certain embodiments, the viral titer is used to estimate the number of stably transduced ES cells that will be produced by a given preparation of packaged virus. In certain embodiments, the feeder cell population in the culture is also infected by the virus.

In certain embodiments, the multiplicity of infection (m.o.i.) is less than about 100. In certain embodiments, the m.o.i. is less than about 50. In certain embodiments, the m.o.i. is less than about 25. In certain embodiments, the m.o.i. is less than about 10. In certain embodiments, the m.o.i. is less than about 5. In certain embodiments, the m.o.i. is less than about 1. In certain embodiments, the m.o.i. is less than about 0.5. In certain embodiments, the m.o.i. is less than about 0.3. In certain embodiments, the m.o.i. is less than about 0.2. In certain embodiments, the m.o.i. is less than about 0.1. In certain embodiments, the m.o.i. is less than about 0.05.

Certain methods of producing retrovirus harboring genetic constructs using retroviral producer cell lines are known in the art. See, e.g., Cone et al. (1984) Proc. Natl. Acad. Sci. USA, 81: 6349-6353; and Miller et al. (1986) Mol. Cell. Biol., 6: 2895-2902. In certain embodiments, retroviral producer cell lines that have been actively cultured for less than about three months are used to produce retrovirus harboring gene trap constructs. In certain embodiments, when retroviral producer cell lines that have been actively cultured for more than about three months are used to produce retrovirus harboring gene trap constructs, the yield of mutated cell clones (made by infection with that retrovirus) from which flanking genomic sequence is acquired decreases. In certain instances, retroviral gene trap constructs recombine with endogenous retroviral sequences present within the mouse genome. Thus, in certain instances, recombination events can accumulate during extended culture and passage. In certain embodiments, such recombination events occur when using the murine packaging cell line GP+E. In certain embodiments, such recombination events occur to an extent that interferes with efficient generation of gene-trapped cell clones mutated by the desired retroviral vectors.

In certain embodiments, engineered retroviral stocks are obtained from retroviral producer cells that have been maintained in active culture for less than about six months. In certain embodiments, the retroviral producer cells used have been maintained in active culture for less than about four months. In certain embodiments, the retroviral producer cells used have been maintained in active culture for less than about three months. In certain embodiments, the retroviral producer cells used have been maintained in active culture for less than about two months. In certain embodiments, the retroviral producer cells used have been maintained in active culture for less than about 1.5 months. In certain embodiments, the retroviral producer cells used have been maintained in active culture for less than about one month. In certain embodiments, the retroviral producer cells used have been maintained in active culture for less than about 21 days. In certain embodiments, the retroviral producer cells used have been maintained in active culture for less than about 14 days. In certain embodiments, the retroviral producer cells used have been maintained in active culture for less than about 10 days. In certain embodiments, the retroviral producer cells used have been maintained in active culture for less than about 5 days. In certain embodiments, the retroviral producer cells used have been maintained in active culture for less than about 3 days. In certain embodiments, the retroviral producer cells used have been maintained in active culture for less than about 2 days. The length of time that a retroviral producer cell has been maintained in active culture is measured from the time a retroviral producer cell is isolated or from the time the retroviral producer cell is thawed and cultured from a frozen stock.

In certain embodiments, retroviral stocks for gene trapping are harvested after the retroviral producer cells are grown to confluence. In certain embodiments, after growing the retroviral producer cells to confluence, the medium is changed, and the retroviral stock is harvested after about 4 to about 48 hours. In certain embodiments, the retroviral stock is harvested after about 8 to about 36 hours after the medium is changed. In certain embodiments, the retroviral stock is harvested after about 12 to about 24 hours after the medium is changed.

In certain embodiments, retroviral producer cells may be stably or transiently transfected with a genetically engineered retroviral genome. In certain embodiments, transiently transfected retroviral producer cells contain a genetically engineered viral genome that is at least partially present episomally.

In certain embodiments, the methods allow for the efficient generation of a collection of gene-trapped cell clones having mutations throughout the genome. In certain embodiments, a collection of mutated cell clones is provided from which mutations in at least about 5,000 different genes have been characterized by identifying one or more regions of genomic DNA sequence flanking the integration site of the gene trap construct. In certain embodiments, a collection of mutated cell clones is provided from which mutations in at least about 7,000 different genes have been characterized. In certain embodiments, a collection of mutated cell clones is provided from which mutations in at least about 10,000 different genes have been characterized. In certain embodiments, a collection of mutated cell clones is provided from which mutations in at least about 15,000 different genes have been characterized. In certain embodiments, a collection of mutated cell clones is provided from which mutations in at least about 20,000 different genes have been characterized. In certain embodiments, a collection of mutated cell clones is provided from which mutations in at least about 25,000 different genes have been characterized. In certain embodiments, a collection of mutated cell clones is provided from which mutations in at least about 30,000 different genes have been characterized. In certain embodiments, the collection of mutated cell clones are mutated ES cell clones.

The following examples are provided for illustrative purposes only and are not to be construed as limiting the present invention in any way.

### 6.0. EXAMPLES

### 6.1. Production of Certain Gene Trapped ES Cells

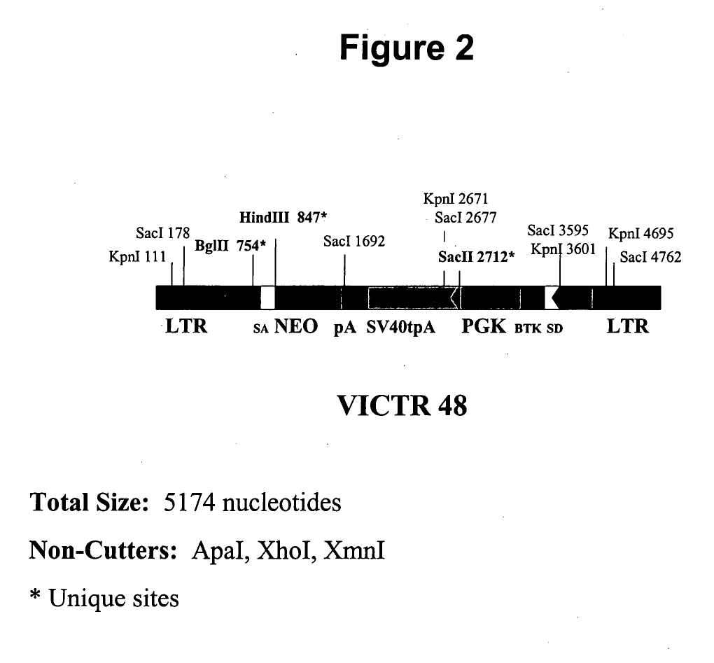

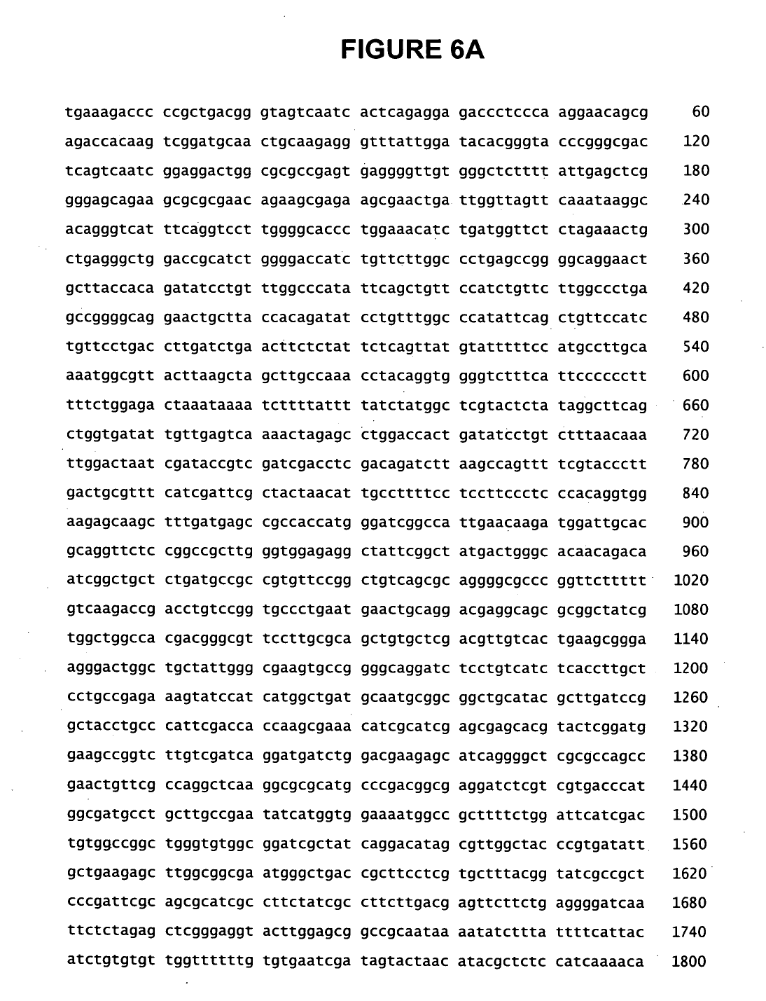

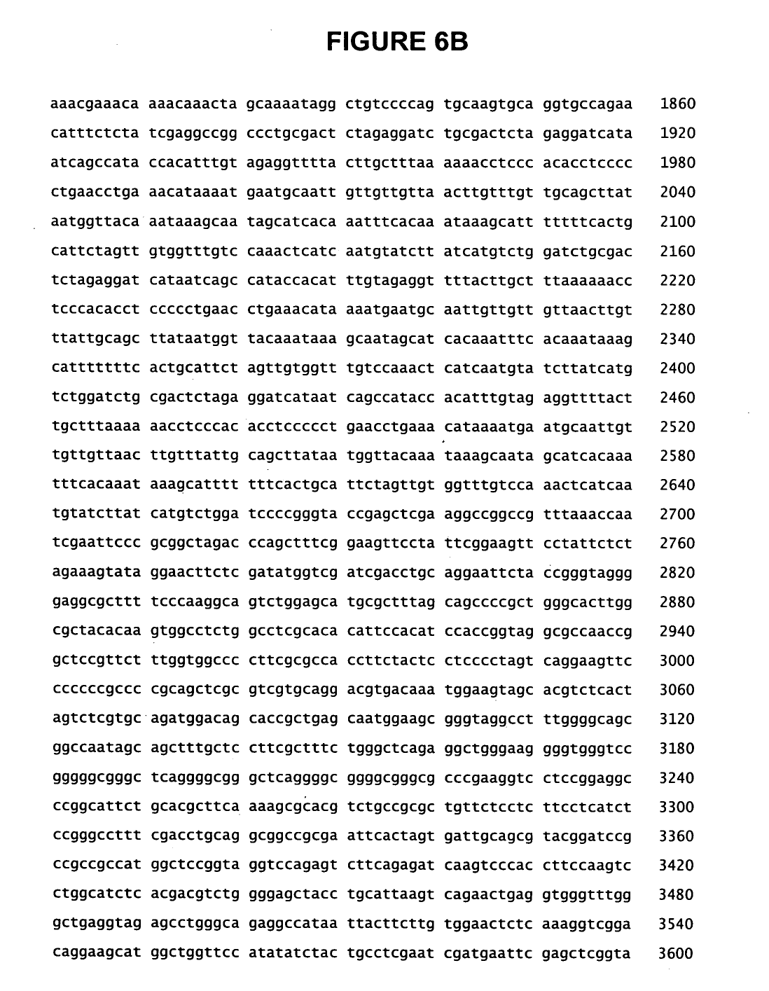

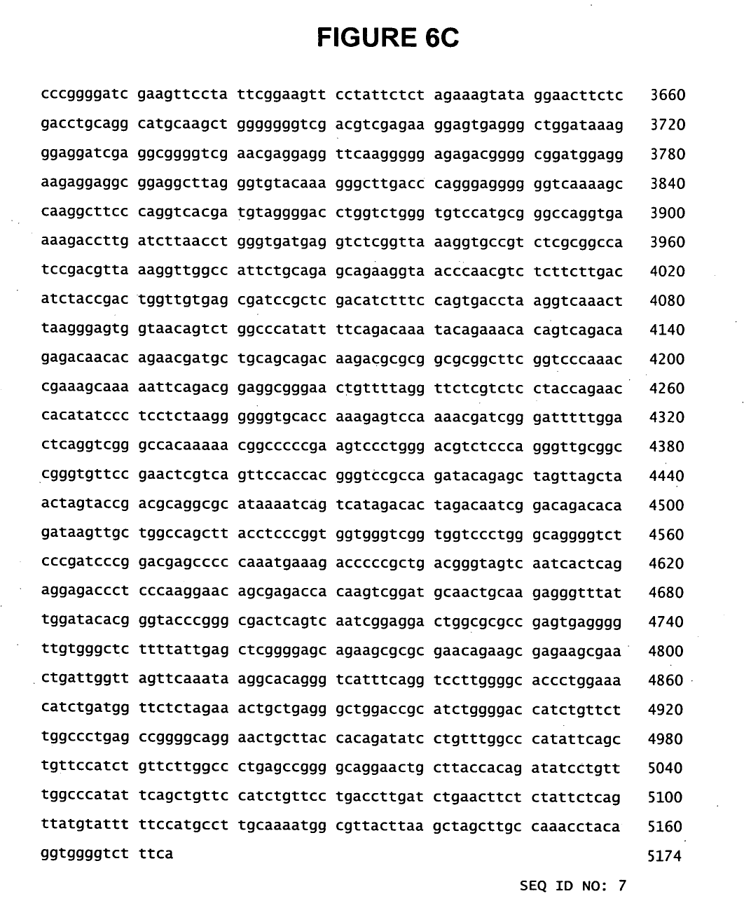

Embryonic stem cells (Lex-1 cells derived from murine strain A129-Sv/Ev) were mutated by infection with the retroviral gene trapping construct VICTR48 at a m.o.i. of approximately 0.3 according to the method described in Zambrowicz et al. (1998) Nature, 392: 608-11. A schematic representation of VICTR48 is shown in FIG. 2. The sequence of VICTR48 is shown in FIGS. 6A-C (SEQ ID NO: 7). Approximately 95 percent of the ES clones that stably integrate the proviral form of the construct were predicted to contain a single integration event. After selecting for ES clones that stably incorporated the construct using G418 selection, the selected cells were seeded onto irradiated feeder cells (SNL cells, which stably express LIF) and cultured to confluence. The confluent wells were subsequently split 1-to-3 into three 96 well plates and again allowed to grow to confluence. Two of the resulting plates are cryogenically preserved and the third plate is processed as follows.

### 6.2. Analysis of Gene Trapped ES Cells

Remove media and rinse cells twice with 100 μl PBS. Add 50 μl lysis solution (50 mM Tris pH 7.5, 50 mM EDTA pH 8.0, 100 mM NaCl, 1% SDS, and 2 mg/ml Proteinase K) to each well, seal the plate, and incubate the plate at 65° C. overnight. Add 150 μl 95% ethanol to each well and let stand at room temperature for 2 hours. Aspirate supernatant and wash with 150 μl 70% ethanol. Aspirate 70% ethanol wash and allow wells to dry for approximately 2 hours at room temperature. Add 200 μl TE (10 mM Tris pH 8.0, 1 mM EDTA) to each well and let stand at room temperature overnight to gently rehydrate genomic DNA. Transfer 10 μl of each genomic DNA to each of six new 96-well plates for endonuclease digestion.

Prepare six digestion mixes, one each for the six different restriction enzymes or restriction enzyme combinations, which are BamHI, EcoRI, HindIII, NcoI, BamHI/BglII, and EcoRI/MfeI. Each digestion mix contains 0.125 units/μl of restriction enzyme in the 1× reaction buffer recommended by the manufacturer, with 1×BSA. For each digestion mix, add 30 μl digestion mix to each well of the digestion plate. Seal the digestion plates and incubate them at 37° C. for 4 hours to overnight. Digestion reactions containing restriction enzymes that cannot be heat inactivated are incubated overnight. The digestion plates are then incubated at 65° C. for 20 minutes.

Add 110 μl ligation mix (2.7 units/μl. (final concentration in each reaction) of T4 DNA ligase in manufacturer's recommended 1× reaction buffer) to each well of each digestion plate. Seal the plates and incubate them at 15° C. overnight. Approximately 10 μl of each ligation product is then added to wells of a new 96-well plate for use as a PCR template.

Sequencing template is generated by adding 40 μl round 1 PCR mix to each well. The round 1 PCR mix contains 0.05 units/μl (final concentration in each reaction) Taq DNA polymerase in manufacturer's recommended 1×PCR buffer, 1.5 mM MgCl₂, 200 nM each DNTP, 1 M Betaine, and 0.1 pmol/μl each round 1 primer. Round 1 primers are:

(SEQ ID NO:1)

forward: 5′ TGAGTCAAAACTAGAGCCTGGACC 3′,

(SEQ ID NO:2)

reverse: 5′ AGTTCGCTTCTCGCTTCTGTTCG 3′.

Plates are sealed and cycled on MJ thermocyclers. Round 1 PCR is as follows. The DNA is denatured at 95° C. for 2 min and pre-annealed at 80° C. for 3 min. The plates are then subjected to 15 cycles of 94° C. for 30 seconds followed by 3 minutes annealing. The annealing temperature in the first cycle is at 70° C., and then the annealing temperature is reduced by 0.5° C. per subsequent cycle. The plates are then subjected to 20 cycles of 94° C. for 30 seconds followed by annealing at 62° C. The annealing time for the first cycle is 3 minutes, and then the annealing time is increased by 1 second per cycle.

Approximately 2 μl of the product from the round 1 PCR reaction is transferred to a new plate and used as a round 2 PCR template. For round 2, 23 μl of round 2 PCR mix is added to each well. The round 2 PCR mix contains 0.05 units/μl (final concentration in each reaction) Taq DNA polymerase in manufacturer's recommended 1×PCR buffer, 1.5 mM MgCl₂, 200 nM each dNTP, 1 M Betaine, and 0.1 pmol/μl each round 2 primer. Round 2 primers are:

(SEQ ID NO:3)

forward: 5′ AAATTGGACTAATCGATACCGTCG 3′,

(SEQ ID NO:4)

reverse: 5′ GAGTGATTGACTACCCGTCAGCG 3′

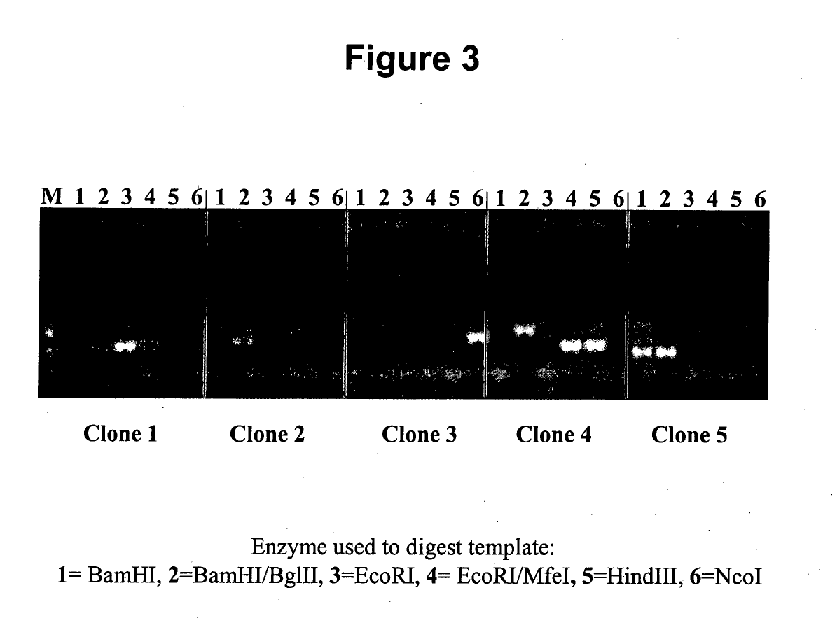

The plates are sealed and then cycled on MJ thermocyclers as described above for Round 1. FIG. 3 shows exemplary IPCR products for five gene-trapped ES cell clones using the six restriction enzymes discussed above. In that experiment, the combination of BamH I and Bgl II resulted in PCR product for four of the five clones.

Approximately 1 μl of the round 2 PCR product is transferred to a new 96-well plate and 9 μl sequencing mix (⅛ of a standard Applied Biosystems Big Dye Version 1.1 reaction) is added to each well. The template is sequenced using 1 μM of the round 2 reverse primer (SEQ ID NO:4). For sequencing, the plates are subject to 25 PCR cycles of 94° C. for 45 seconds, 52° C. for 15 seconds, and 60° C. for 2 minutes.

The completed sequencing reactions are cleaned prior to electrophoresis using prepared 96-well sephadex plates (Millipore MultiScreen 96-well plates containing hydrated Sephadex G-50 Fine from AmershamBiosciences), which are centrifuged at 2000 rpm for 5 minutes. The eluted reactions are dried and then resuspended in 8 μl water. The resuspension is then loaded on an ABI Prism® 3700 DNA Analyzer (Applied Biosystems) with RunModule ‘MGCore’.

The resulting sequences are deposited in FASTA format into a local database available for BLAST searching. In addition, sequence data for the clones are aligned by BLAST to obtain a single consensus sequence that can be used to map each mutation to the mouse genome.

### 6.3. Results of Analysis using BglII/BamHI and IPCR

Exemplary results show that employing a single enzyme pair (BglII/BamHI) yields sequence (average length greater than about 250 base pairs) in about 60 percent of the ES cell clones that are screened using this method. In the exemplary results, other enzymes produce sequence as follows: NcoI produces sequence in 20 percent of the ES cell clones screened; HindIII produces sequence in 23 percent of the ES cell clones screened; and EcoRI produces sequence in 14 percent of the ES cell clones screened. The net yield of ES cell clones for which sequence is obtained in the exemplary results is approximately 85 percent (some ES cell clones produce sequence for more than one enzyme and/or enzyme combination).

### 6.4. Removal of LTR Cryptic Splice Donor

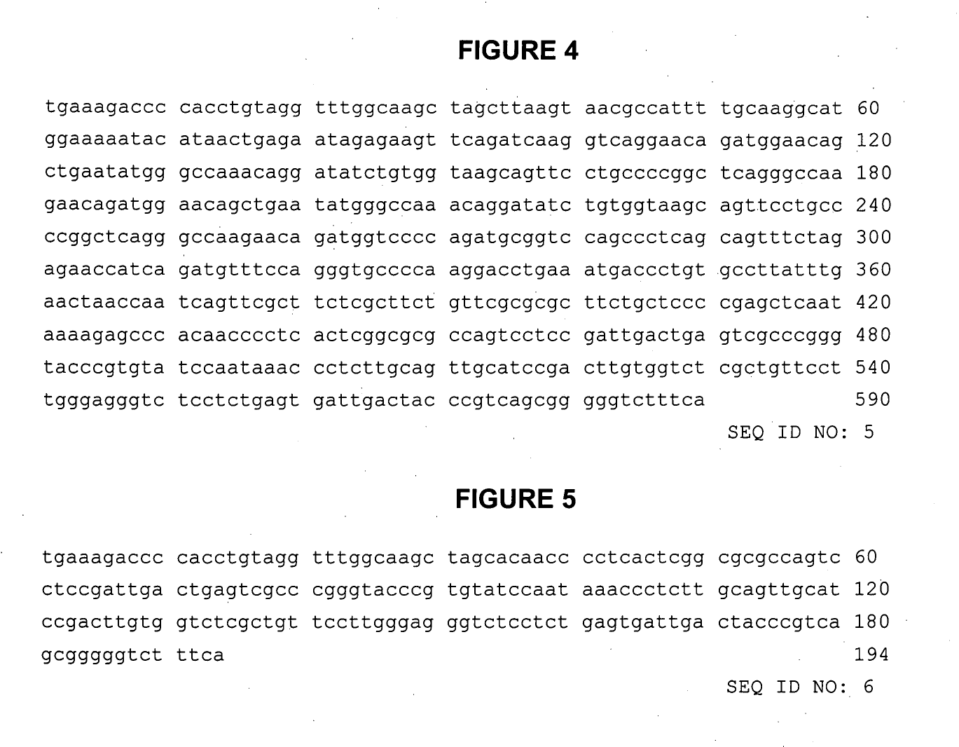

The Moloney murine leukemia virus long terminal repeat (LTR) may be modified by deleting at least a portion of the enhancer region (FIG. 4, SEQ ID NO:5). The Moloney murine leukemia virus LTR may be further modified by deleting a cryptic splice donor within the LTR, in addition to at least a portion of the enhancer region (FIG. 5, SEQ ID NO:6). In certain instances, the enhancer and/or cryptic splice donor may function in the reverse orientation of normal retroviral transcription In certain embodiments, by deleting the cryptic reverse-orientation splice donor, the fidelity of obtaining gene trap events within genes is enhanced.

The present invention is not to be limited in scope by the specific embodiments described herein, which are intended only as illustrations of certain aspects of certain embodiments of the invention, and functionally equivalent methods and components are within the scope of the invention. Indeed, various modifications, in addition to those shown and described herein will be apparent to those skilled in the art from the foregoing description. Such modifications are intended to fall within the scope of the appended claims. All cited publications, patents, and patent applications are herein incorporated by reference in their entirety for any purpose.

1. A process for producing a collection of individually characterized insertionally mutated mammalian cell clones comprising:
a) infecting mammalian cells with a retroviral gene trap construct at a multiplicity of infection of less than 5;
b) selecting mammalian cell clones stably incorporating an integrated proviral form of said retroviral gene trap construct; and
c) identifying in vitro a region of genomic DNA adjacent to the integrated proviral form of said retroviral gene trap construct, wherein the identifying does not involve a reverse transcriptase reaction.

2. The process according to claim 1 wherein said multiplicity of infection is less than 1.

3. The process according to claim 2 wherein said multiplicity of infection is less than 0.5.

4. The process according to claim 3 wherein said identifying is by sequencing at least 50 bases of genomic DNA adjacent to the integrated proviral form of said retroviral gene trap construct.

5. The process according to claim 3 wherein a collection of at least 10,000 different mammalian cell clones is selected.

6. The process according to claim 5, wherein the collection of at least 10,000 different mammalian cell clones comprises at least 10,000 different mammalian cell cones that each have an integrated proviral form of said retroviral gene trap construct in a different gene.

7. The process according to claim 1, wherein the identifying comprises an inverse polymerase chain reaction (IPCR).

8. The process according to claim 7, wherein the inverse polymerase chain reaction comprises at least one polymerase selected from Pfu, Taq, Isis, Vent, Pwo, Phusion, and Tth.

9. A collection of individually characterized insertionally mutated mammalian cell clones produced by the process of claim 1.

10. The process according to claim 7, wherein the inverse polymerase chain reaction does not comprise Phusion polymerase.
